(19) United States
(12) Patent Application Publication
(10) Pub. No.: US 2024/0315540 A1
(43) Pub. Date: Sep. 26, 2024

(54) ENDOSCOPIC PROBE, ENDOSCOPE AND METHOD OF SCANNING CONTROL

(71) Applicant: SHENZHEN METALENX TECHNOLOGY CO.,LTD, Shenzhen (CN)

(72) Inventors: Rui ZHU, Shenzhen (CN); Jian ZHU, Shenzhen (CN); Chenglong HAO, Shenzhen (CN); Fengze TAN, Shenzhen (CN)

(73) Assignee: SHENZHEN METALENX TECHNOLOGY CO.,LTD, Shenzhen (CN)

(21) Appl. No.: 18/732,501

(22) Filed: Jun. 3, 2024

Related U.S. Application Data
(63) Continuation of application No. PCT/CN2022/143190, filed on Dec. 29, 2022.

(30) Foreign Application Priority Data
Jan. 5, 2022 (CN) 202210008890.5

(51) Int. Cl. A61B 1/045; A61B 1/00; A61B 1/06; A61B 1/07

(52) U.S. Cl. CPC A61B 1/045; A61B 1/00105; A61B 1/0016; A61B 1/063; A61B 1/07

(57) ABSTRACT

An endoscopic probe is provided, and the endoscopic probe includes: an optic fiber and a metalens. The optic fiber includes: a signal input fiber core, a signal output fiber core and a coating; the signal input fiber core is used to transport an input laser signal. The metalens includes: a translucent substrate, a plurality of nanostructures arranged on the one side of the surface of the substrate. The plurality of nanostructures are arranged in array and attached to a distal surface of the signal input fiber core, so as to focus the input laser signal on an inner surface of tissues to be detected; and the signal output fiber core is used for transporting a laser signal reflected through the inner surface of the tissue to be detected, so as to obtain an image of the inner surface of the tissue to be detected after the reflected laser signal processing.

### CROSS-REFERENCE TO RELATED APPLICATION

This application is a continuation of International Patent Application of PCT application serial No. PCT/CN2022/143190, filed on Dec. 29, 2022, which claims the benefit of priority from China Application No. 202210008890.5, filed on Jan. 5, 2022. The entirety of each of the above mentioned patent applications is hereby incorporated by reference herein and made a part of this specification.

### TECHNICAL FIELD

The present disclosure relates to the field of an endoscopy, in particular to an endoscopic probes, an endoscope and methods of scanning control.

### BACKGROUND

Endoscopy can enter and visualize the interior of a patient's cavity for the purpose of diagnosis and/or treatment. For example, during surgery or examination, an endoscope can be inserted into the patient, and the device can pass through the endoscope to the tissue parts identified for diagnosis and/or treatment.

The conventional endoscope shown in FIG. 1 has an image sensor, objective lens, light guiding window, jaw outlet, nozzle and auxiliary water supply hole. It has a complex structure, large size and high manufacturing cost.

As an important part of the endoscope, the structural complexity, size and manufacturing cost of the endoscopic probe will inevitably affect the applications of the endoscope. How to better reduce the structural complexity, size and manufacturing cost of the endoscopic probe is an urgent problem to be solved by those skilled in the field.

### SUMMARY

In view of the above technical problems, an endoscopic probes, an endoscope and methods of scanning control are provided according to embodiments of the present disclosure, so as to overcome the problems in the related art.

According to one aspect of the present disclosure, an endoscopic probe is provided, and the endoscopic probe includes: an optic fiber and a metalens. The optic fiber includes: a signal input fiber core, a signal output fiber core and a coating; the signal input fiber core is used to transport an input laser signal; and the metalens includes: a translucent substrate, a plurality of nanostructures arranged on the one side of the surface of the substrate, and the plurality of nanostructures are arranged in array and attached to a distal surface of the signal input fiber core, so as to focus the input laser signal on an inner surface of a tissue to be detected; and the signal output fiber core is used for transporting a laser signal reflected through the inner surface of the tissue to be detected, so as to obtain an image of the inner surface of the tissue to be detected after the reflected laser signal processing.

Optionally, the signal output fiber core includes: a plurality of circle fiber cores; the plurality of circle fiber cores are set in the diametrical direction of the signal input fiber core.

Optionally, the endoscopic probe can be rotated on the center of the signal input fiber core and is able to move together with an attached endoscope body while rotating.

Optionally, a near-end of the endoscopic probe is connected to a rotary joint.

Optionally, the plurality of nanostructures are adhered to the distal surface of the signal input fiber core.

Optionally, the diametrical dimension of the distal surface of the signal input fiber core is equal to the diametrical dimension of an array formed by the plurality of nanostructures. Optionally, an edge of the substrate is aligned with the edge of the distal surface of the coating.

Optionally, the a protective film is provided on the side of the plurality of nanostructures attached to the distal surface.

Optionally, for each of the plurality of nanostructures, there are six nanostructures located at different vertices of one regular hexagon, and one nanostructure is located at the center of the regular hexagon.

Optionally, for each of the plurality of nanostructures, there are four nanostructures located at different vertices of one square, and one nanostructure is located at the center of the square.

Optionally, the plurality of nanostructures is made by one of the following: titanium oxide, silicon nitride, molten quartz, alumina, gallium nitride, gallium phosphate, amorphous silicon, and crystalline silicon.

Optionally, the metalens coated with the protective film and the distal surface of the coating are adhered by a glue;
an edge of the substrate is aligned with an edge of the coating in the process of adhering, and the array formed by the plurality of nanostructures is aligned with the signal input fiber core.

According to the other aspect of the present disclosure, an endoscope is provided, and the endoscope includes the endoscopic probe provided by the one aspect of the present disclosure.

Optionally, the endoscopic probe is in detachable connection with an endoscope body.

Optionally, the endoscope body includes: a single photon avalanche diode, an image display device and a micromotor; the single photon avalanche diode is used for collecting signals; the micromotor is used for rotating.

Optionally, the endoscopic probe is connected to the endoscope body by a rotary joint.

According to another aspect of the present disclosure, a scanning control method for the endoscopic probe is provided, and the endoscopic probe includes: an optic fiber and a metalens. And the optic fiber comprises: a signal input fiber core, a signal output fiber core and a coating; the signal input fiber core is used to transport an input laser signal. The metalens includes: a translucent substrate, a plurality of nanostructures arranged on the same surface of the substrate, and the plurality of nanostructures are arranged in array and attached to a distal surface of the signal input fiber core, so as to focus the input laser signal on an inner surface of a tissue to be detected; and the signal output fiber core is used for transporting a laser signal reflected through the inner surface of the tissue to be detected, so as to obtain an image of the inner surface of the tissue to be detected after the reflected laser signal processing. And the signal output fiber core includes: a plurality of circle fiber cores; the plurality of circle fiber cores are set in the diametrical direction of the signal input fiber core. And the scanning control method for the endoscopic probe includes: controlling the simultaneous movement of the endoscopic probe rotating around the signal input fiber core as a center; when any of the signal output fiber core is connected to a photodetector at the near-end of the endoscopic probe, the laser signal transported by the signal output fiber core is outputted.

Optionally, the scanning control method further includes:
the signal input fiber core is used for transporting the laser signal, and the circle fiber core is used for collecting the reflected laser signal from the tissue to be detected.

Optionally, a number of signals collected by the photodetector during per unit time is as following:

$$\\ F = \frac{2 π}{nw}$$

where, n is a number of signal output fiber core, w is the rotation speed of the endoscopic probe, and F is the frame rate of the output image.

According to the solutions of the present disclosure, because the metalens is light, cheap, easy to install, and the method of metalens production has high capacity, the structural complexity, size and manufacturing cost of the endoscopic probe are reduced under the use of the metalens. And the one-time use of the endoscopy provided by present disclosure can improve the safety. One endoscopic probe is also used to simultaneously achieve illumination and signal collecting, and it can further reduce the size and improve safety.

### BRIEF DESCRIPTION OF THE DRAWINGS

The present disclosure may be better understood by reference to the description given below in combination with the drawings, where the same or similar drawing markings are used in all the drawings to represent the same or similar components. The drawings are included in the specification along with the following detailed description and form part of the specification, and to further illustrate the preferred embodiments of the disclosure and explain the principles and advantages of the disclosure.

FIG. 1 shows a basic structure of an endoscope known to the prior art.

FIG. 2A shows a sectional view of the endoscopic probe according to an embodiment of the present disclosure.

FIG. 2B shows a cross-sectional view of a metalens in an endoscopic probe according to an embodiment of the present disclosure.

FIG. 3 shows a schematic diagram of the laser signal transporting pathway in the endoscopic probe according to an embodiment of the present disclosure.

FIG. 4 shows a schematic fiber cross section view in an endoscopic probe according to an embodiment of the present disclosure.

FIG. 5 shows a layout of a metasurface structure unit of a metalens in an endoscopic probe according to an embodiment of the present disclosure.

FIG. 6A to FIG. 6B show a schematic view of a metalens in an endoscopic probe according to an embodiment of the present disclosure.

FIG. 7 shows a schematic view of gastric tissue endoscopy based on an endoscopic probe according to an embodiment of the present disclosure.

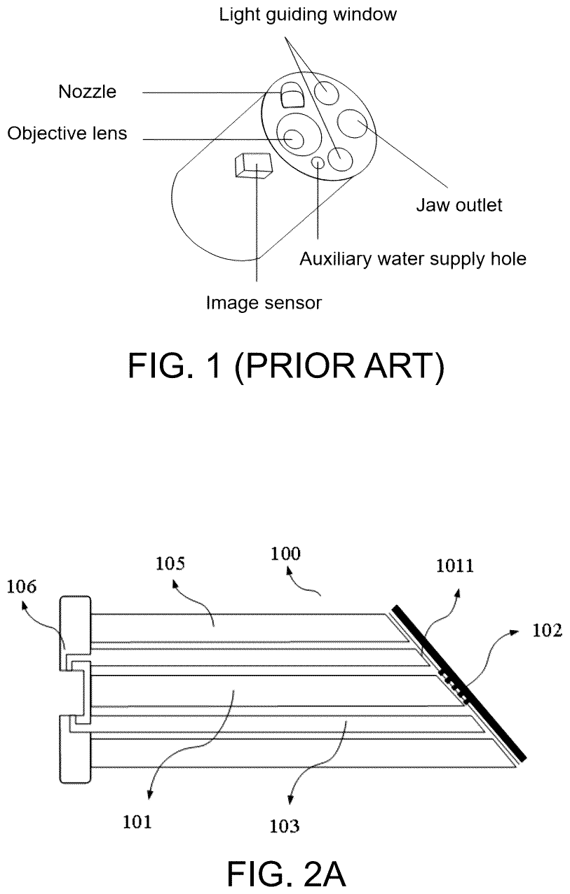

Those skilled in the art should understand that the elements in the accompanying drawings are shown only for simplicity and clarity, and are not necessarily drawn to scale. For example, the dimensions of some elements in the drawings may be enlarged relative to other elements in order to help improve the understanding of the embodiments of the disclosure.

### DETAILED DESCRIPTION OF DISCLOSURE EMBODIMENTS

The technical scheme in the embodiment of this disclosure will be clearly and completely described in combination with the attached drawings of the present disclosure method. Obviously, the implementation method described clearly is only part of the implementation method of the present disclosure, rather than all of the implementation methods. Based on the mode of implementation in this disclosure, all other modes of implementation obtained by persons skilled in the field without making creative labor fall within the scope of protection in this disclosure.

It should be understood that terms used in the present disclosure, such as “central”, “longitudinal”, “transverse”, “length”, “width”, “thickness”, “upper”, “lower”, “front”, “rear”, “left”, “right”, “vertical”, “horizontal”, “top”, “bottom”, “interior”, “exterior”, “clockwise”, “counterclockwise” which are intended to indicate orientational or positional relationships based on the accompanying drawings are only for the purpose of describing the present disclosure conveniently and simply, and are not intended to indicate or imply a particular orientation, a structure and an operation in a particular orientation of the device or element referred to herein, and thus are not to be interpreted as a limitation to the present disclosure.

In addition, terms “first” and “second” are used for descriptive purposes, and are not intended to indicate or imply relative importance or implicitly indicate the quantity of the indicated technical features. Therefore, features defined by “first” or “second” may explicitly or implicitly include one or more of these features. In the description of the present disclosure, “plurality” or “multiple” means that there are two or more of these features, unless otherwise explicitly and specifically defined.

In this disclosure, the word “exemplary” is used to mean “used as an example, illustration or explanation”. Any embodiment described in this disclosure is not necessarily interpreted as more preferred or advantageous than other embodiments. In order to enable anyone skilled in the art to implement and use the present disclosure, the following description is given. In the following description, the details are listed for the purpose of the interpretation. It should be understood that those skilled in the art may recognize that the present disclosure may also be implemented without using these specific details. In other examples, the known structure and process will not be elaborated to avoid unnecessary details to obscure the description of this disclosure. Therefore, the present disclosure is not intended to be limited to the embodiment shown, but is consistent with the broadest scope consistent with the principles and characteristics disclosed in the present disclosure.

### Endoscopic Probe

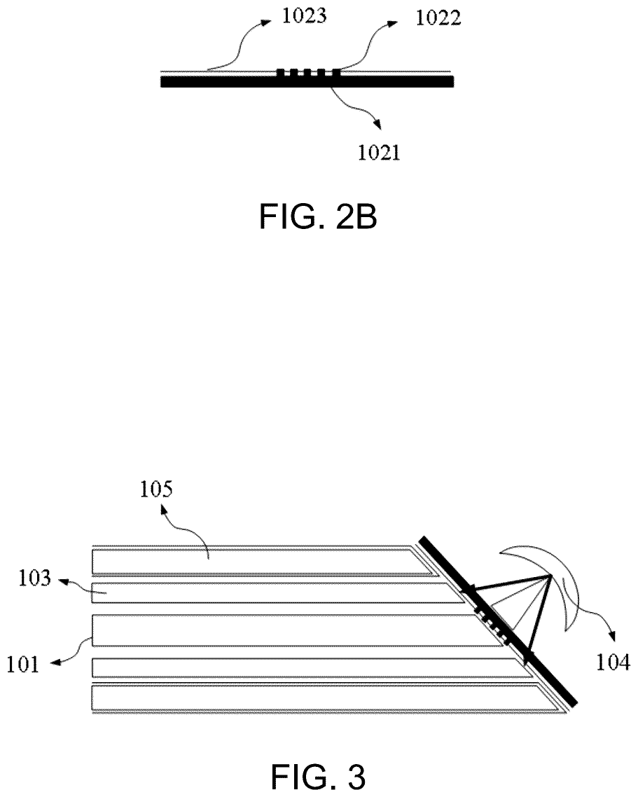

An endoscopic probe can be used for imaging the inner surface of biological living tissue. According to the embodiment of the present disclosure, an endoscopic probe 100 is provided, as shown in FIG. 2A and FIG. 3, the endoscopic probe 100 includes: an optic fiber and a metalens 102. The optic fiber includes: a signal input fiber core 101, a signal output fiber core 103 and a coating 105, the signal input fiber core 101 is used to transport an input laser signal. And the metalens 102 includes: a translucent substrate 1021 and a plurality of nanostructures 1022 arranged on the one side of the surface of the substrate, where the plurality of nanostructures 1022 are arranged in array and attached to a distal surface 1011 of the signal input fiber core 101, so as to focus the input laser signal on an inner surface 104 of a tissue to be detected; and the signal output fiber core 103 is used for transporting a laser signal reflected through the inner surface 104 of the tissue to be detected, so as to obtain an image of the inner surface 104 of the tissue to be detected after the reflected laser signal processing.

As shown in FIG. 2A and FIG. 2B, the endoscopic probe 100 mainly includes a signal input fiber core 101 for inputting signals, a signal output core 103 for outputting signals and a metalens 102 attached to the signal input fiber core 101 and the distal surface of the signal output fiber core 103. The signal input fiber core 101 and the signal output fiber fiber core 103 are coated with the coating 105 in the length direction, in one embodiment, the edge of the substrate 1021 is aligned with the edge of the distal surface 1011 of the coating, and plurality of nanostructures 1022 is adhered to the distal surface 1011 of the signal input fiber core 101. It is understood that the nanostructures 1022 may be attached to the distal surface 1011 in other ways than gluing, and the edge of the substrate 1021 may not be aligned with the edge of the distal surface 1011 of the coating 105, which does not serve as a limitation of the present disclosure. In this disclosure, the distal end is the end far away from the operator during the use of the endoscope. In this embodiment, the end close to the inner surface 104 of the tissue to be detected. And the near end is closer to the operator during the use of the endoscope, that is, the farther end from the inner surface 104 of the tissue to be detected.

The cross-section diagram of the metalens 102 is shown in FIG. 2B, the size of the substrate 1021 may be the same as the size of the coating 105. In one embodiment, the size of the substrate 1021 may be the same as the distal surface of the optical fiber. In one embodiment, a protective film 1023 is provided on the side of the plurality of nanostructures 1022 attached to the distal surface 1011. The protective film 1023 is a filler material, and the filler material is air or a transparent or translucent material at the working waveband. The metalens 102 coated with the protective film 1023 and the distal surface of the coating 105 are adhered by a glue. An edge of the substrate 1021 is aligned with an edge of the coating 105 in the process of adhering, and the array formed by the plurality of nanostructures 1022 is aligned with the signal input fiber core 101. In one embodiment, the diametrical dimension of the distal surface 1011 of the signal input fiber core 101 is equal to the diametrical dimension of an array formed by the plurality of nanostructures 1022.

In one embodiment, a near-end of the endoscopic probe 100 is connected to a rotary joint 106. And the endoscopic probe 100 is connected to an endoscope body with the rotary joint 106. The endoscope body mainly includes: a single photon avalanche diode, an image display device and a micromotor. The single photon avalanche diode is used for collecting signals, and the micromotor is used for rotating. In this embodiment, the connection method between the endoscopic probe and the endoscope body is as an example to describe. Those skilled in the art can understand that the endoscopic probe may be in detachable connection with the endoscope body in other ways, so that different endoscopic probes can be replaced before each using. For example, using different endoscopic probes for different patients can utilize one-time use to improve safety.

FIG. 3 shows a specific pathway of transporting signals. As shown in FIG. 3, the laser signals transporting through the signal input fiber core 101 pass through the metalens 102, and then the laser signals focus to the inner surface 104 of the tissue to be detected. After being reflected through the inner surface 104, the laser signals are collected by the signal output fiber core 103 and transported to the inner-end of the signal output fiber core 103, so as to perform a signal processing. While the endoscopic probe 100 is rotating, the endoscopic probe 100 moves on the inner surface 104 of the tissue to be detected, and obtains all the image information of the inner surface 104.

Because the metalens is light, simple, cheap and the method of metalens production has high capacity, the structural complexity, size and manufacturing cost of the endoscopic probe are reduced under the use of the metalens. At the same time, one endoscopic probe is used to perform both functions of illumination and collecting signal, further reducing the size of the endoscopic probe and improving safety.

### Scanning Control Method

According to an embodiment of the present disclosure, a scanning control method for the endoscopic probe is provided. The endoscopic probe 100 includes: an optic fiber and a metalens 102. And the optic fiber includes: a signal input fiber core 101, a signal output fiber core 103 and a coating 105 and the signal input fiber core is used to transport an input laser signal. The metalens 102 includes: a translucent substrate 1021 and a plurality of nanostructures 1022 arranged on the same surface of the substrate 1021, and the plurality of nanostructures 1022 are arranged in array and attached to a distal surface 1011 of the signal input fiber core 101. In this way, the input laser signal may focus on an inner surface 104 of a tissue to be detected. And the signal output fiber core 103 is used for transporting a laser signal reflected through the inner surface 104 of the tissue to be detected, so as to obtain an image of the inner surface 104 of the tissue to be detected after the reflected laser signal processing. And the signal output fiber core 103 includes: a plurality of circle fiber cores, and the plurality of circle fiber cores are set in the diametrical direction of the signal input fiber core 101. The scanning control method for the endoscopic probe includes:

Controlling the simultaneous movement of the signal output fiber core 103 rotating around the signal input fiber core 101 as a center, and when any of the signal output fiber core 103 is connected to a photodetector at the near-end of the endoscopic probe 100, the laser signal transported by the signal output fiber core 103 is outputted.

The micromotor for rotating is connected only to the endoscopic probe 100. While the endoscopic probe 100 rotating, the endoscope body will not rotate, witch means that only the endoscopic probe 100 will rotate. The endoscopic probe 100 will move forward together with the endoscope body. After arriving at the inner surface 104 of the tissue to be detected, the endoscopic probe 100 moves on the inner surface 104 of the tissue to be detected for performing the detection. During the using, the endoscope body will not rotate and only the endoscopic probe 100 will rotate, so that the operational difficulty is reduced and the operational safety is improved.

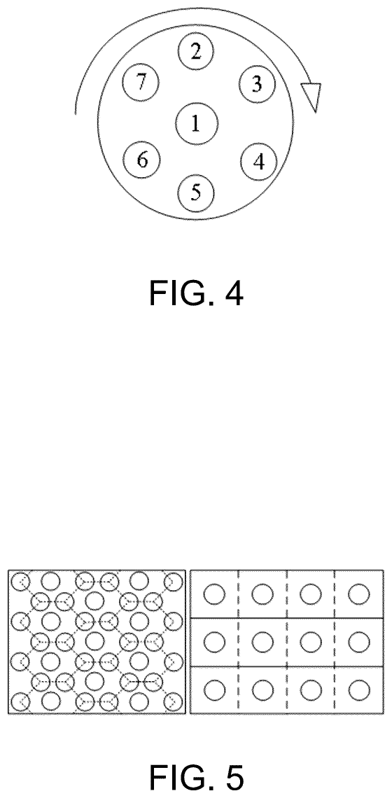

The signal input fiber core 101 at the center is used for transporting the laser signal to the tissue to be detected, and the circle fiber core is used for collecting the reflected laser signal from inner surface 104 of the tissue to be detected. As shown in FIG. 4, the signal input fiber core 101 is used for transporting signals to the tissue to be detected, and the signal output signal 103 is used for transporting the reflected laser signal to the photodetector, and the reflected laser signal contains the information of the tissue to be detected, and the photodetector is connected to the rotary joint 106. When any of the signal output fiber core is connected to a photodetector by the rotary joint 106, the laser signal transported by the signal output fiber core is outputted. Then the photodetector performs the received laser signals processing, for example, the photodectector may transfer the laser signals into electrical signals.

In on embodiment, assumed the rotation is along the clockwise direction of the optical fiber, that is, the endoscopic probe is rotating in the order of 2-3-4-5-6-7 as shown in FIG. 4. When any circle fiber core is contacted or connected to the photodetector, the laser signals within the signal output fiber core 103 can be detected. And the circle fiber cores are set in the diametrical direction of the signal input fiber core 101. A number of signals collected by the photodetector during per unit time is as equation (1):

$$\\ \begin{matrix}F = \frac{2 π}{nw} & ( 1 )\end{matrix}$$

Where n is a number of signal output fiber core, w is the rotation speed of the endoscopic probe, and F is the frame rate of the output image.

Assuming the length of the surface of the tissue to be detected in the horizontal direction is L, the time that required to scan the entire surface of the tissue to be detected is shown in equation (2):

$$\\ \begin{matrix}t = \frac{L}{v} & ( 2 )\end{matrix}$$

And m is the number of the rotations of the endoscopic probe 100, and is shown in equation (3) as following:

$$\\ \begin{matrix}m = \frac{wL}{2 π v} & ( 3 )\end{matrix}$$

Inputting equation (3) into equation (2), the frame rate F can be obtained in equation (4) as following:

$$\\ \begin{matrix}F = \frac{L}{nvm} & ( 4 )\end{matrix}$$

According to the equation (4), improving the frame rate and obtaining a more clear image may be utilized by reducing the number of signal output fiber cores and the rotation of the endoscopic probe, or by reducing the movement speed in the horizontal direction of the endoscopic probe when the tissue size to be detected is certain.

### Metalens

Metalens is a kind of metasurface. The metasurface is sub-wavelength artificial nano-structured film, and has unit cells on its surface. The unit cells may be used for modulating the phase of the incident light. And the unit cells include full dielectric medium or plasma nanoantennas which can modulate the phase, amplitude or polarization of lights.

In the present disclosure, the nanostructures 1022 are full dielectric medium structures, which have a high transmittance at the wavebands of the visible light. And the plurality of nanostructures 1022 are made by one of the following: titanium oxide, silicon nitride, molten quartz, alumina, gallium nitride, gallium phosphate, amorphous silicon, and crystalline silicon.

In addition, the nanostructures 1022 are arranged in array. The metalens includes: a plurality of unit cells. Each unit cell is composed a nanostructure and the unit cells are arranged in array. The shape of the unit cell may be a regular hexagon and/or a square. As shown in FIG. 5, an arrangement diagram of the regular hexagon unit cells and an arrangement diagram of the square unit cells is provided.

If the shapes of the unit cells are regular hexagons, for each of the plurality of nanostructures 1022, there are six nanostructures located at different vertices of one regular hexagon, and one nanostructure is located at the center of the regular hexagon. If the shapes of the unit cells are squares, for each of the plurality of nanostructures 1022, there are four nanostructures located at different vertices and the plurality of nanostructures are located at the vertices and the center of the unit cell, respectively. In other words, the plurality of nanostructures 1022 are located at the vertices and the center of the unit cell, respectively. In this arrangement, the metalens 102 may have the least number of nanostructures 1022, and the performance of the metalens 102 may also meet requirements.

In one embodiment, the thickness of the substrate 1021 may be greater than or equal to 0.1 mm and less than 2 mm, such as, the thickness of the substrate 1021 may be 0.1 mm, 0.5 mm, 1 mm, 1.5 mm, 2 mm.

The thickness of the overall structure formed by the plurality of nanostructures 1022 of this embodiment is of the micron level, so the nanostructures 1022 on the substrate 1021 can approximate to a one-planar structure. Optionally, the thickness of the structure that formed by the plurality of nanostructures 1022 is less than or equal to 50 μm, such as, 1.5 μm, 5 μm, 10 μm, 1.5 μm, 20 μm, 25 μm, 30 μm, 35 μm, 40 μm, 45 μm, 50 μm. It is further noted that in the embodiment of the present disclosure, the thickness of the metalens 102 is the sum of the thickness of the overall structure formed by the plurality of nanostructures 1022 and the thickness of the substrate 1021. It should be noted that the substrate 1021 is only a structure that supports the plurality of nanostructures 1022, and the material of the substrate 1021 may be the same or different from the material of the nanostructure 1022.

Exemplary, the substrate 1021 may be made of quartz glass or crystalline silicon, and it should be understood that the substrate 1021 may be made of other materials.

In addition, the shape of the metalens 102 may be determined by the shape of the substrate 1021, which may be a regular shape like a round, a square, a regular polygon, or an irregular shape. Exemplary, the substrate 1021 is circular, and the shape of the metalens 102 is circular. Exemplary, the substrate 1021 is square, and the shape of the metalens 102 is square.

In the present embodiment, the working waveband of the metasurface is near-infrared. The nanostructures 1022 may be filled with air or the transparent or translucent materials at the working wavebands. It should be noted that the absolute value of the difference between the refractive index of the transparent or translucent materials and the refractive index of the nanostructures 1022 should be greater than or equal to 0.5. The nanostructure 1022 is symmetrical about a first axis and a second axis. And the first axis and the second axis are vertical, and the first axis and the second axis are perpendicular to the height direction of the nanostructure 1022, respectively. The first axis and the second axis pass through the center of the nanostructure 1022, and are parallel to the horizontal plane.

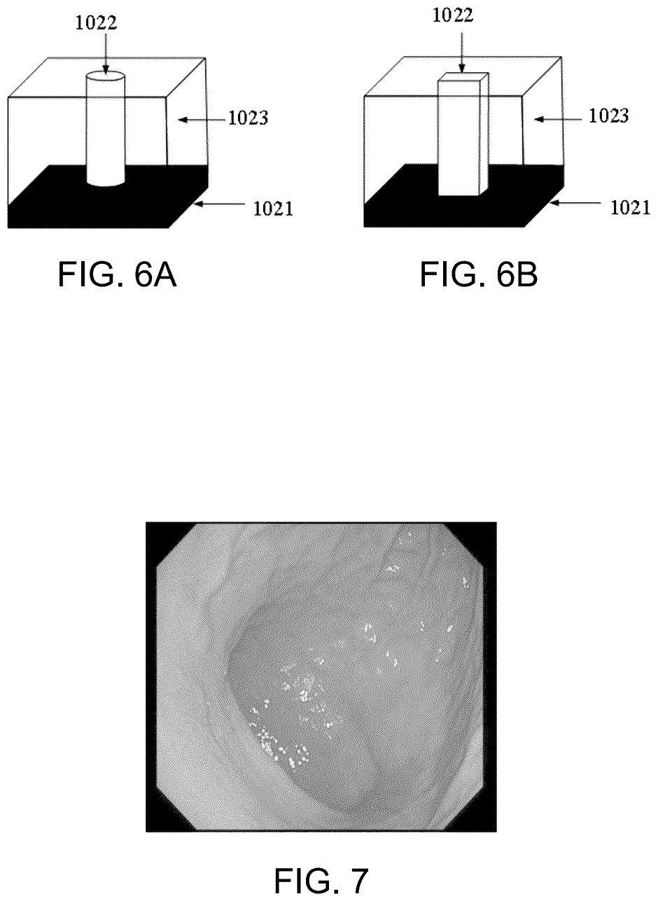

The nanostructures 1022 may be a polarization-dependent structure, such as nanopillar fins and nano elliptical columns shown in FIG. 6A and FIG. 6B, those structures apply a Pancharatnam-Berry Phase to the incident light. In addition, the nanostructures 1022 may be a polarization-independent structure, suchlike circle nanopillars and square nanopillars, and those structures apply a Propagation Phase to the incident light.

### Endoscope

According to the embodiment of the present disclosure, an endoscope is provided, including the endoscopic probe 100 in the above embodiments. The endoscopic probe 100 is in detachable connection with an endoscope body. In one optional embodiment, the endoscopic probe is connected to the endoscope body by a rotary joint. The micromotor for rotating may be only connect to the endoscopic probe 100. During the rotation of the endoscopic probe 100, the endoscopic body will not rotate. The endoscopic probe 100 and the endoscopic body are moving forward along with the endoscope body. After arriving at the inner surface 104 of the tissue to be detected, the endoscopic probe 100 moves on the inner surface 104 of the tissue to be detected for detection during the rotation. The endoscope based on the endoscopic probe 100 keeps the endoscope body motionless during using, and only the endoscopic probe 100 rotates, which can reduce the difficulty of operation and using. Meanwhile the safety of the endoscope is improved.

### Scenarios for Using

In using, the endoscopic probe is connected to the endoscope body by a rotary joint. By using remote or far field operations, the endoscopic probe moves together with the endoscope body to insert into the gastric tissue inside patient, and the micromotor makes the endoscopic probe rotate. While the endoscope body is rotating, the endoscope body moves on the inner surface of the gastric tissue to image the inner surface of gastric tissue. After imaging, the endoscopic probe may be removed by rotating the rotary joint in a counterclockwise direction. Before imaging another patient, a new endoscopic probe can be installed to the endoscope body by the rotary joint.

FIG. 7 shows a schematic diagram of internal endoscopic imaging of gastric tissue based on endoscopic probes according to an embodiment of the present disclosure. In this embodiment, the length of the stomach is about 25 cm, the number of signal output fiber core, namely the circle fiber core is 8, and the frame rate of the output image is about 15 frames per second.

The ordinary technician in the field should understand that the discussion of the above embodiment is exemplary only and is not intended to imply that the scope of the disclosure (including the claims) is limited to these examples; under the thinking of the disclosure, the above embodiment or the technical characteristics of the different embodiments can also be combined, and there are many other changes in the different aspects of the disclosure as described above, to state that they are not provided in detail.

It should be emphasized that the term “include/contain” refers to the presence of features, elements, steps or components when used herein, but does not exclude the presence or addition of one or more other features, elements or components. The terms “first”, “second” involving ordinal numbers do not indicate the order of implementation, or importance of the features, elements, steps or components defined by these terms, but are merely used to identify these features, elements, steps or components for the purpose of clarity.

Although the disclosure is described in accordance with a limited number of embodiments, benefiting from the above description, those skilled in the art understand that within the scope of the herein described disclosure. Furthermore, it should be noted that the language used in this specification is chosen primarily for the purpose of readability and instruction, and not for the purpose of explaining or defining the subject matter of the disclosure. Therefore, without departing from the scope and spirit of the attached claim, many modifications and changes are obvious to the ordinary technicians in the technical field. For the scope of the disclosure, the disclosure is illustrative but not restrictive, and the scope of the disclosure is defined by the attached claim.

What is claimed is:

1. An endoscopic probe, wherein the endoscopic probe comprises: an optic fiber and a metalens;
wherein, the optic fiber comprises: a signal input fiber core, a signal output fiber core and a coating;
the signal input fiber core is used to transport an input laser signal;
the metalens comprises: a translucent substrate, a plurality of nanostructures arranged on the one side of the surface of the substrate, wherein, the plurality of nanostructures are arranged in array and attached to a distal surface of the signal input fiber core, so as to focus the input laser signal on an inner surface of a tissue to be detected; and
the signal output fiber core is used for transporting a laser signal reflected through the inner surface of the tissue to be detected, so as to obtain an image of the inner surface of the tissue to be detected after the reflected laser signal processing.

2. The endoscopic probe according to claim 1, wherein the signal output fiber core comprises: a plurality of circle fiber cores;
the plurality of circle fiber cores are set in the diametrical direction of the signal input fiber core.

3. The endoscopic probe according to claim 2, the endoscopic probe can be rotated on the center of the signal input fiber core and is able to move together with an attached endoscope body while rotating.

4. The endoscopic probe according to claim 1, wherein a near-end of the endoscopic probe is connected to a rotary joint.

5. The endoscopic probe according to claim 1, wherein the plurality of nanostructures are adhered to the distal surface of the signal input fiber core.

6. The endoscopic probe according to claim 1, wherein the diametrical dimension of the distal surface of the signal input fiber core is equal to the diametrical dimension of an array formed by the plurality of nanostructures.

7. The endoscopic probe according to claim 1, wherein an edge of the substrate is aligned with the edge of the distal surface of the coating.

8. The endoscopic probe according to claim 1, wherein a protective film is provided on the side of the plurality of nanostructures attached to the distal surface.

9. The endoscopic probe according to claim 1, wherein for each of the plurality of nanostructures, there are six nanostructures located at different vertices of one regular hexagon, and one nanostructure is located at the center of the regular hexagon.

10. The endoscopic probe according to claim 1, wherein for each of the plurality of nanostructures, there are four nanostructures located at different vertices of one square, and one nanostructure is located at the center of the square.

11. The endoscopic probe according to claim 1, wherein the plurality of nanostructures is made by one of the following: titanium oxide, silicon nitride, molten quartz, alumina, gallium nitride, gallium phosphate, amorphous silicon, and crystalline silicon.

12. The endoscopic probe according to claim 8, wherein the metalens coated with the protective film and the distal surface of the coating are adhered by a glue;
an edge of the substrate is aligned with an edge of the coating in the process of adhering, and the array formed by the plurality of nanostructures is aligned with the signal input fiber core.

13. An endoscope, wherein the endoscope comprises the endoscopic probe of claim 1.

14. The endoscope according to claim 13, wherein the endoscopic probe is in detachable connection with an endoscope body.

15. The endoscope according to claim 14, wherein the endoscope body comprises: a single photon avalanche diode, an image display device and a micromotor;
the single photon avalanche diode is used for collecting signals;
the micromotor is used for rotating.

16. The endoscope according to claim 14, wherein the endoscopic probe is connected to the endoscope body by a rotary joint.

17. A scanning control method for an endoscopic probe, wherein the endoscopic probe comprises an optic fiber and a metalens;
wherein, the optic fiber comprises: a signal input fiber core, a signal output fiber core and a coating;
the signal input fiber core is used to transport an input laser signal;
the metalens comprises: a translucent substrate, a plurality of nanostructures arranged on the same surface of the substrate, wherein, the plurality of nanostructures are arranged in array and attached to a distal surface of the signal input fiber core, so as to focus the input laser signal on an inner surface of a tissue to be detected; and
the signal output fiber core is used for transporting a laser signal reflected through the inner surface of the tissue to be detected, so as to obtain an image of the inner surface of the tissue to be detected after the reflected laser signal processing;
wherein, the signal output fiber core comprises: a plurality of circle fiber cores;
the plurality of circle fiber cores are set in the diametrical direction of the signal input fiber core;
the scanning control method for the endoscopic probe comprises:
controlling the simultaneous movement of the endoscopic probe rotating around the signal input fiber core as a center; wherein, when any of the signal output fiber core is connected to a photodetector at the near-end of the endoscopic probe, the laser signal transported by the signal output fiber core is outputted.

18. The scanning control method for the endoscopic probe according to claim 17, wherein the scanning control method further comprises:
the signal input fiber core is used for transporting the laser signal, and the circle fiber core is used for collecting the reflected laser signal from the tissue to be detected.

19. The scanning control method for the endoscopic probe according to claim 17, wherein a number of signals collected by the photodetector during per unit time is as following:
$$\\ F = \frac{2 π}{nw}$$
wherein, n is a number of signal output fiber core, w is the rotation speed of the endoscopic probe, and F is the frame rate of the output image.
